(19) United States
(12) Patent Application Publication
(10) Pub. No.: US 2026/0272953 A1
(43) Pub. Date: Sep. 17, 2026

(54) CANNABINOID FORMULATIONS

(71) Applicant: DSM IP Assets B.V., Maastricht (NL)

(72) Inventors: Ahmed BESHEER, Kaiseraugst (CH); Elger FUNDA, Kaiseraugst (CH); Thomas LINDEMANN, Kaiseraugst (CH); Christian SCHAEFER, Kaiseraugst (CH); Christiane SCHWEIGGERT, Kaiseraugst (CH); Thomas ZWICK, Kaiseraugst (CH)

(21) Appl. No.: 19/163,688

(22) PCT Filed: Mar. 14, 2024
(86) PCT No.: PCT/EP2024/056836

§ 371 Date: Sep. 9, 2025

(30) Foreign Application Priority Data
Mar. 14, 2023 (EP) 23161772.1
Mar. 14, 2023 (EP) 23161774.7

(51) Int. Cl. A61K 31/00; A61K 9/16; A61K 9/20

(52) U.S. Cl. CPC A61K 31/658; A61K 9/1611; A61K 9/1617; A61K 9/1623; A61K 9/1652; A61K 9/1658; A61K 9/1694; A61K 9/2009; A61K 9/2013; A61K 9/2018; A61K 9/2027; A61K 9/2054; A61K 9/2059; A61K 9/2063

(57) ABSTRACT

The present invention relates to a new formulation comprising a high amount of at least one cannabinoid, wherein the cannabinoid is in a non-crystalline form.

The present invention relates to a new formulation comprising a high amount of at least one cannabinoid, wherein the cannabinoid is in a non-crystalline form.

Cannabinoids are several structural classes of compounds found in the cannabis plant primarily and most animal organisms or as synthetic compounds. At least 113 different cannabinoids have been isolated from the cannabis plant up to today.

There are many different cannabinoids and terpenoids present in a sample of cannabis, however only the main cannabinoids have been linked to pharmacological activity so far.

The main cannabinoid types are:
cannabidiol (CBD), tetrahydrocannabinol (THC), cannabinol (CBN), cannabigerol (CBG), cannabichromene (CBC), cannabicitran (CBT), cannabinodivarin (CBV), cannabiripsol (CBR), hexahydrocannabinol (HHC), delta-9-tetrahydrocannabiphoral (THCP), tetrahydrocannabivarin (THCV), endocannabinoid (anandamide) and endocannabinoid (2-AG).

Cannabinoids are known to have health benefits, which are shown in medical studies such as i.e., lowering blood pressure, reducing inflammation, preventing relapse in drug and alcohol addiction, treating anxiety disorders, treating gastrointestinal (GI) disorders, and preventing seizures.

Cannabinoids can be administered in multiple ways, including by inhaling cannabis smoke or vapor, orally e.g., in the form of a tablet or capsule, and as an aerosol spray into the cheek.

Crystalline cannabinoids are found to have low oral bioavailability. For instance, it is estimated that only 6% of an orally administered dose of crystalline cannabinoid was bioavailable under fasting conditions. On the other hand, lipid-dissolved cannabinoid has been reported to exhibit an approximately 4-fold increased bioavailability.

A problem in regard of formulating cannabinoids, particularly in high dosage forms, is the poor water solubility as well as the limited solubility of cannabinoids in various oils. This makes it challenging to provide liquid or solid dosage forms containing high amounts of one or more cannabinoids, in particular in non-crystalline form for improved bioavailability.

Therefore, the goal of the present invention was to provide a formulation, wherein at least one cannabinoid is formulated in a high amount, and wherein the at least one cannabinoid is (and remains upon storage) in a substantially non-crystalline form.

Furthermore, this formulation should be usable to produce further application forms (such as powders, tablets, capsules etc), which are also storage stable.

Surprisingly it was found that using a MCT oil (Medium-Chain Triglyceride) and at least one cannabinoid in a specific ratio, a stable emulsion with a high amount of at least one cannabinoid (in a non-crystalline form) is obtained, which in addition exhibited a particularly good bioavailability compared to the use of other oils.

This liquid formulation (emulsion/dispersion) can then be used as such or be transformed into a solid formulation (such as powder), wherein the at least one cannabinoid is contained in amounts of up to approximately 45 wt.-%, while being in a substantially non crystalline, i.e., better bioavailable form.

Therefore, the present invention relates to a liquid formulation (LF) comprising
(i) 5-85 weight-% (wt-%), based on the total weight of the liquid formulation, of at least one cannabinoid, and
(ii) 2 to 25 wt-%, based on the total weight of the liquid formulation, of at least one MCT oil, and
(iii) 1 to 50 wt-%, based on the total weight of the liquid formulation, of at least one emulsifier, and
(iv) 12 to 55 wt-%, based on the total weight of the liquid formulation, of water,
wherein at least 50 wt-%, based on the total weight of the cannabinoid, of the at least one cannabinoid is in a non-crystalline form and
the ratio of the at least one cannabinoid to the at least one MCT oil is 2:1 to 4:1.

In the context of the present invention, all percentages of a formulation always add up to 100 wt.-%.

In the context of the present invention, the preferred cannabinoids are chosen from the group consisting of cannabidiol, tetrahydrocannabinol, cannabinol, cannabigerol, cannabichromene, cannabicitran, cannabinodivarin, cannabiripsol, hexahydrocannabinol, delta-9-tetrahydrocannabiphoral, tetrahydrocannabivarin, endocannabinoid and endocannabinoid.

More preferred cannabinoids are chosen from the group consisting of cannabidiol, tetrahydrocannabinol, cannabinol, cannabigerol, cannabichromene, cannabicitran, cannabinodivarin, cannabiripsol, hexahydrocannabinol and delta-9-tetrahydrocannabiphoral.

Especially preferred cannabinoids are chosen from the group consisting of cannabidiol, tetrahydrocannabinol, cannabinol and cannabigerol (most preferred is cannabidiol).

Most preferred in all embodiments of the present invention is the use of cannabidiol.

Therefore, the present invention relates to a liquid formulation (LF1), which is the liquid formulation (LF), wherein at least one cannabinoid is chosen from the group consisting of cannabidiol, tetrahydrocannabinol, cannabinol, cannabigerol, cannabichromene, cannabicitran, cannabinodivarin, cannabiripsol, hexahydrocannabinol, delta-9-tetrahydrocannabiphoral, tetrahydrocannabivarin, endocannabinoid and endocannabinoid.

Therefore, the present invention relates to a liquid formulation (LF1′), which is the liquid formulation (LF), wherein at least one cannabinoid is chosen from the group consisting of cannabidiol, tetrahydrocannabinol, cannabinol, cannabigerol, cannabichromene, cannabicitran, cannabinodivarin, cannabiripsol, hexahydrocannabinol and delta-9-tetrahydrocannabiphoral.

Therefore, the present invention also relates to a liquid formulation (LF1″), which is the liquid formulation (LF), wherein at least one cannabinoid is chosen from the group consisting of cannabidiol, tetrahydrocannabinol, cannabinol and cannabigerol (most preferred is cannabidiol).

The amount of the of the at least one cannabinoid in the liquid formulation according to the present invention is 5-85 wt-%, based on the total weight of the liquid formulation.

Preferably, the amount of at least one cannabinoid in the liquid formulation according to the present invention is 10-85 wt-%, based on the total weight of the liquid formulation. More preferred, the amount of at least one cannabinoid in the liquid formulation according to the present invention is 10-80 wt-%, based on the total weight of the liquid formulation.

Even more preferred, the amount of at least one cannabinoid in the liquid formulation according to the present invention is 15-80 wt-%, based on the total weight of the liquid formulation.

Most preferably, in all embodiments of the present invention, the amount of the at least one cannabinoid in the liquid formulation according to the present invention is selected in the range from 7.5 to 50 wt.-%, in particular from 10 to 50 wt.-%, most in particular from 15-50 wt-%. Further suitable ranges are selected in the range from 15 to 45 wt.-%, from 15 to 40 wt.-%, from 15 to 35 wt.-%, from 15 to 30 wt.-%, from 20 to 45 wt.-%, from 20 to 40 wt.-%, from 20 to 35 wt.-%, from 20 to 30 wt.-%, from 25 to 45 wt.-%, from 25 to 40 wt.-%, from 30 to 45 wt.-%, from 30 to 40 wt.-%, from 35 to 45 wt.-% as well as from 35 to 40 wt.-% based, on the total weight of the liquid formulation.

Therefore, the present invention also relates to a liquid formulation (LF2), which is the liquid formulation (LF), (LF1), (LF1′) or (LF1″), wherein the amount of the of the at least one cannabinoid in the formulation according to the present invention is 10-85 wt-%, based on the total weight of the liquid formulation.

Therefore, the present invention also relates to a liquid formulation (LF2′), which is the liquid formulation (LF), (LF1), (LF1′) or (LF1″), wherein the amount of the of the at least one cannabinoid in the formulation according to the present invention is 10-80 wt-%, based on the total weight of the liquid formulation.

Therefore, the present invention also relates to a liquid formulation (LF2″), which is the liquid formulation (LF), (LF1), (LF1′) or (LF1″), wherein the amount of the of the at least one cannabinoid in the formulation according to the present invention is 15-80 weight-%, based on the total weight of the liquid formulation.

As stated above, at least 50 wt-%, based on the total weight of the cannabinoid, of the one cannabinoid is in a non-crystalline form.

More preferred, in all embodiments of the present invention at least 55 wt-%, based on the total weight of the cannabinoid, of the one cannabinoid is in a non-crystalline form.

Even more preferred, in all embodiments of the present invention at least 60 wt-%, based on the total weight of the cannabinoid, of the one cannabinoid is in a non-crystalline form.

Even more preferred, in all embodiments of the present invention at least 70 wt-%, based on the total weight of the cannabinoid, of the one cannabinoid is in a non-crystalline form.

Even more preferred, in all embodiments of the present invention at least 75 wt-%, based on the total weight of the cannabinoid, of the one cannabinoid is in a non-crystalline form.

Even more preferred, in all embodiments of the present invention at least 80 wt-%, based on the total weight of the cannabinoid, of the one cannabinoid is in a non-crystalline form.

Even more preferred, in all embodiments of the present invention at least 85 wt-%, based on the total weight of the cannabinoid, of the one cannabinoid is in a non-crystalline form.

Even more preferred, in all embodiments of the present invention at least 90 wt-%, based on the total weight of the cannabinoid, of the one cannabinoid is in a non-crystalline form.

Even more preferred, in all embodiments of the present invention at least 95 wt-%, based on the total weight of the cannabinoid, of the one cannabinoid is in a non-crystalline form.

Even more preferred, in all embodiments of the present invention at least 98 wt-%, based on the total weight of the cannabinoid, of the one cannabinoid is in a non-crystalline form.

Even more preferred, in all embodiments of the present invention at least 99 wt-%, based on the total weight of the cannabinoid, of the one cannabinoid is in a non-crystalline form.

Even more preferred, in all embodiments of the present invention 100 wt-%, based on the total weight of the cannabinoid, of the one cannabinoid is in a non-crystalline form.

In all embodiments of the present invention the at least one cannabinoid is most advantageously in a substantially non-crystalline form, even more preferably dissolved in the at least one MCT oil.

In the context of the present invention, the term ‘crystalline’ is understood to mean a coherent or non-coherent part of one or more components with homogeneous physical properties, in particular a homogeneous melting range and a long-range order. It is well understood that crystals are solid, i.e. in a solid state of aggregation.

In the context of the present invention, the term ‘non-crystalline’ is understood to mean a cohesive or non-cohesive part of one or more components with homogeneous physical properties, in particular without any homogeneous melting range and long-range order.

In all embodiments of the present invention most advantageously, the non-crystalline cannabinoid is liquid, i.e. is present in a liquid (e.g. dissolved) state of aggregation, such as in particular dissolved in the ate least one MCT oil. Even more preferably, the non-crystalline cannabinoid is not or not substantially amorphous but dissolved.

In the context of the present invention, the term ‘long range order’ is understood to mean a regular and periodic spacing of molecules or atoms (here of the at least one cannabinoid) in a crystalline solid. Accordingly, the exact position of a few molecules or atoms can advantageously be used to determine the position of all molecules or atoms in a crystalline solid.

The content of the crystalline form of the at least one cannabinoid in the liquid and solid formulation according to the present invention can be measured using commonly known methods, such as differential scanning calorimetry (DSC).

In the context of the present invention DSC was used to determine the amount of crystalline form of the at least one cannabinoid in the liquid respectively solid formulation according to the present invention.

Thus, the in all embodiments according to the present invention, preferably the degree of crystallinity (in %) is determined by DSC using the respective crystalline cannabinoid as external standard.

The DSC method as used in the present invention is carried out as follows:

Liquid or powder (solid) formulations (ca. 8-10 mg) were placed in aluminum pans and hermetically sealed. Empty pans were used as a reference samples. For the investigation of the cannabinoid bulk material (i.e. the respective crystalline cannabinoid), about 1-2 mg of sample was used (external standard). Each sample was heated from 10° C. at 10 C/min to 90° C. and then cooled down to 10° C. at 10 C/min. Upon heating, for the crystalline cannabinoid compound the thermal transition peak is determined (observed), which for CBD was determined to be at ca. 68° C., which was attributed to the melting of the cannabidiol crystals. By analyzing the DSC thermograms and measuring the peak areas, the degree of crystallinity of a sample can then be determined, as the peak area of the DSC peak corresponds to the amount of heat released during crystallization/heat consumed during melting and is proportional to the degree of crystallinity of a sample. To determine the % of crystallinity, the peak areas of the samples are referenced against the peak area of the crystalline cannabinoid. The amount of crystalline cannabinoid present in the respective sample can then be calculated, which is then used to determine the degree of crystallinity (%), based on known total amount of cannabinoid present in the sample.

Therefore, the present invention also relates to a liquid formulation (LF3), which is the liquid formulation (LF), (LF1), (LF1′), (LF1″), (LF2), (LF2′) or (LF2″), wherein at least 55 wt-%, based on the total weight of the cannabinoid, of the one cannabinoid is in a non-crystalline form.

Therefore, the present invention also relates to a liquid formulation (LF4), which is the liquid formulation (LF), (LF1), (LF1′), (LF1″), (LF2), (LF2′) or (LF2″), wherein at least 60 wt-%, based on the total weight of the cannabinoid, of the one cannabinoid is in a non-crystalline form.

Therefore, the present invention also relates to a liquid formulation (LF5), which is the liquid formulation (LF), (LF1), (LF1′), (LF1″), (LF2), (LF2′) or (LF2″), wherein at least 65 wt-%, based on the total weight of the cannabinoid, of the one cannabinoid is in a non-crystalline form.

Therefore, the present invention also relates to a liquid formulation (LF6), which is the liquid formulation (LF), (LF1), (LF1′), (LF1″), (LF2), (LF2′) or (LF2″), wherein at least 70 wt-%, based on the total weight of the cannabinoid, of the one cannabinoid is in a non-crystalline form.

Therefore, the present invention also relates to a liquid formulation (LF7), which is the liquid formulation (LF), (LF1), (LF1′), (LF1″), (LF2), (LF2′) or (LF2″), wherein at least 75 wt-%, based on the total weight of the cannabinoid, of the one cannabinoid is in a non-crystalline form.

Therefore, the present invention also relates to a liquid formulation (LF8), which is the liquid formulation (LF), (LF1), (LF1′), (LF1″), (LF2), (LF2′) or (LF2″), wherein at least 80 wt-%, based on the total weight of the cannabinoid, of the one cannabinoid is in a non-crystalline form.

Therefore, the present invention also relates to a liquid formulation (LF9), which is the liquid formulation (LF), (LF1), (LF1′), (LF1″), (LF2), (LF2′) or (LF2″), wherein at least 85 wt-%, based on the total weight of the cannabinoid, of the one cannabinoid is in a non-crystalline form.

Therefore, the present invention also relates to a liquid formulation (LF10), which is the liquid formulation (LF), (LF1), (LF1′), (LF1″), (LF2), (LF2′) or (LF2″), wherein at least 90 wt-%, based on the total weight of the cannabinoid, of the one cannabinoid is in a non-crystalline form.

Therefore, the present invention also relates to a liquid formulation (LF11), which is the liquid formulation (LF), (LF1), (LF1′), (LF1″), (LF2), (LF2′) or (LF2″), wherein at least 95 wt-%, based on the total weight of the cannabinoid, of the one cannabinoid is in a non-crystalline form.

Therefore, the present invention also relates to a liquid formulation (LF12), which is the liquid formulation (LF), (LF1), (LF1′), (LF1″), (LF2), (LF2′) or (LF2″), wherein at least 98 wt-%, based on the total weight of the cannabinoid, of the one cannabinoid is in a non-crystalline form.

Therefore, the present invention also relates to a liquid formulation (LF13), which is the liquid formulation (LF), (LF1), (LF1′), (LF1″), (LF2), (LF2′) or (LF2″), wherein 100 wt-%, based on the total weight of the cannabinoid, of the one cannabinoid is in a non-crystalline form.

The liquid formulation according to the present comprises at least one MCT oil.

MCT oils are triglycerides with two or three fatty acids having an aliphatic tail of 6-12 carbon atoms, i.e. medium-chain fatty acids (MCFAs). Examples are caproic acid, caprylic acid, capric acid and lauric acid. Natural sources of these oils are i.e. palm kernel oil or coconut oil.

MCT oils can be bought from a variety of suppliers.

Therefore, the present invention also relates to a liquid formulation (LF14), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12) or (LF13), wherein the MCT oil is chosen from the group consisting of caproic acid, caprylic acid, capric acid and lauric acid.

The liquid formulation according to the present invention comprises 2 to 25 wt-%, based on the total weight of the liquid formulation, of at least one MCT oil.

Preferably the amount of the at least one MCT oil is selected in the range from 3 to 20 wt-%, based on the total weight of the liquid formulation.

Therefore, the present invention also relates to a liquid formulation (LF15), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13) or (LF14), wherein the content of the is 3 to 20 wt-%, based on the total weight of the liquid formulation.

Most preferably, in all embodiments of the present invention, the amount of the at least one MCT oil in the liquid formulation according to the present invention is selected in the range from 5 to 20 wt.-%, such as in particular from 5 to 15 wt.-%, based on the total weight of the liquid formulation.

As stated above the ratio of the at least one cannabinoid to The at least one MCT oil is 2:1 to 4:1 in the liquid formulation according to the present invention.

Preferably, the ratio of the at least one cannabinoid to The at least one MCT oil is 2:1 to 3.5:1.

More preferred, the ratio of the at least one cannabinoid to The at least one MCT oil is 2.2:1 to 3.5:1.

Another preferred ratio of the at least one cannabinoid to the at least one MCT oil is 3 to 4, such as most in particular the ratio is about 4.

Therefore, the present invention also relates to a liquid formulation (LF16), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), or (LF15), wherein the ratio of the at least one cannabinoid to the at least one MCT oil is 2:1 to 3.5:1.

Therefore, the present invention also relates to a liquid formulation (LF16′), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), or (LF15), wherein the ratio of the at least one cannabinoid to the at least one MCT oil is 2.2:1 to 3.5:1.

Furthermore, the liquid formulations according to the present invention comprise at least one emulsifier. Any commonly known and used emulsifier can be used. A single emulsifier as well as a mixture of emulsifiers can be used.

Suitable emulsifiers are modified (food) starches, Vitamin E TPGS, ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia (=gum arabic), modified gum acacia, TIC gum, flaxseed gum, ghatti gum, tamarind gum and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof).

Preferred emulsifiers in all embodiments of the present invention are modified (food) starches (OSA starch), Vitamin E TPGS, polysaccharide gums, gelatine (bovine, fish, pork, poultry), and plant proteins as well as mixtures thereof.

The starches can be modified physically and chemically. Pregelatinized starches are examples of physically modified starches. Acidic modified, oxidized, cross-linked, starch esters, starch ethers and cationic starches are examples of chemically modified starches. A particular suitable starch in all embodiments of the invention is octenyl succinate starch (OSA starch) which is e.g. commercially available as Cleargum at Roquette or Capsul HS from Ingredion.

Therefore, the present invention also relates to a liquid formulation (LF17), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16) or (LF16′), wherein the at least one emulsifier is chosen from the group consisting of modified (food) starches, Vitamin E TPGS (CAS no 9002-96-4), ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia (=gum arabic), modified gum acacia, TIC gum, flaxseed gum, ghatti gum, tamarind gum and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof).

Therefore, the present invention also relates to a liquid formulation (LF17′), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16) or (LF16′), wherein the at least one emulsifier is chosen from the group consisting of modified (food) starches, Vitamin E TPGS, polysaccharide gums, gelatine (bovine, fish, pork, poultry) and plant proteins. In all embodiments of the present invention, the use of gelatine is preferred.

The liquid formulation according to the present invention comprises 1 to 50 wt-%, based on the total weight of the liquid formulation, of at least one emulsifier.

Preferably, the liquid formulation according to the present invention comprises 5 to 50 wt-%, based on the total weight of the liquid formulation, of at least one emulsifier.

Therefore, the present invention also relates to a liquid formulation (LF18), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16), (LF16′), (LF17) or (LF17′), wherein the amount of the at least one emulsifier is 5 to 50 wt-%, based on the total weight of the liquid formulation.

Most preferably, in all embodiments of the present invention, the amount of the at least one emulsifier in the liquid formulation according to the present invention is selected in the range from 10 to 50 wt.-%, in particular from 15 to 50 wt.-%, such as most in particular from 20 to 50 wt-%. Further suitable ranges are selected from 15 to 45 wt.-%, from 15 to 40 wt.-%, from 15 to 35 wt.-%, from 20 to 45, wt.-%, from 20 to 40 wt.-%, as well as from 20 to 35 wt.-%, based on the total weight of the liquid formulation.

In all embodiments of the present invention preferably the liquid formulation according to the present comprises Vitamin E TPGS and at least one other emulsifier (other than vitamin E TPGS) as this further reduces the particle size of the oil droplets, which is preferred.

Particular suitable emulsifiers in all embodiments according to the present invention is gelatine, a mixture of gelatine and vitamin E TPGS or a mixture of OSA starch and Vitamin E TPGS, preferably in the absence of any other further emulsifiers. Most preferably in all embodiments of the present invention is the use of gelatine, even more preferably in combination with Vitamin E TPGS and most preferably in the absence of any further emulsifiers.

Vitamin E TPGS (TPGS stands for tocopherol polyethylene glycol succinate) also known as yocofersolan (INN) or tocophersolan, is a synthetic water-soluble version of vitamin E. The IUPAC name is α-Hydro-ω-{[4-oxo-4-({(2R)-2,5,7,8-tetramethyl-2-[(4R,8R)-4,8,12-trimethyltridecyl]-3,4-dihydro-2H-1-benzopyran-6-yl}oxy)butanoyl]oxy}poly (oxyethylene). The CAS number is 9002-96-4.

Vitamin E TPGS is the compound of the following formula

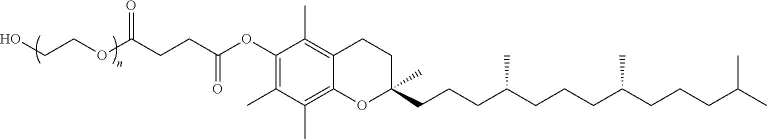

Preferably, in all embodiments of the present invention the liquid formulation according to the present invention comprises 0.1 to 10 wt-%, based on the total weight of the liquid formulation, of Vitamin E TPGS and 0.9 to 40 wt-%, based on the total weight of the liquid formulation, of at least one other emulsifier (other than vitamin E TPGS) such as in particular gelatine or OSA starch, most preferably gelatine.

More preferably in all embodiments of the present invention, the liquid formulation according to the present invention comprises 0.1 to 8 wt-%, based on the total weight of the liquid formulation, of Vitamin E TPGS and 0.9 to 42 wt-%, based on the total weight of the liquid formulation, of at least one other emulsifier (other than Vitamin E TPGS) such as in particular gelatine or OSA starch.

In all embodiments of the present invention preferably, the ratio (w/w) of gelatine respectively OSA starch to Vitamin E TPGS is selected in the range of 90: to 1:1, preferably in the range of 90 to 2.5:1, most preferably in the range of 90:1 to 5:1. Further suitable ranges are from 50:1 to 5:1, from 25:1 to 5:1 and from 10:1 to 5:1.

The liquid formulation according to the present invention comprises 12 to 55 wt-%, based on the total weight of the liquid formulation, of water.

Preferably, the liquid formulation according to the present invention comprises 15 to 55 wt-%, based on the total weight of the liquid formulation, of water.

Preferably, the liquid formulation according to the present invention comprises 20 to 55 wt-%, based on the total weight of the liquid formulation, of water.

Therefore, the present invention also relates to a liquid formulation (LF19), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16), (LF16′), (LF17), (LF17′), (LF18), wherein the amount of water is 15 to 55 wt-%, based on the total weight of the liquid formulation.

Therefore, the present invention also relates to a liquid formulation (LF19′), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16), (LF16′), (LF17), (LF17′), (LF18), wherein the amount of water is 20 to 55 wt-%, based on the total weight of the liquid formulation

Most preferably, in all embodiments of the present invention, the amount of water in the liquid formulation according to the present invention is selected in the range from 15 to 50 wt.-%, in particular from 15 to 45 wt.-%, such as most in particular from 15 to 40 wt-%, based on the total weight of the liquid formulation.

The liquid formulation according to the present invention can comprise optionally at least one further auxiliary agent. Such auxiliary agent can be dyestuffs, thickeners (such as maltodextrin, glucose syrup), fillers, binders, flavours, antioxidants, pH buffer.

The content of these auxiliary agents can be up to 40 wt-%, based on the total weight of the liquid formulation.

Therefore, the present invention also relates to a liquid formulation (LF20), which is the liquid formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16), (LF16′), (LF17), (LF17′), (LF18), (LF19) or (LF19′), wherein the liquid formulation comprises at least one auxiliary agent chosen from the group consisting of dyestuffs, thickener (such as maltodextrin, (dried) glucose syrup), fillers, binders, flavours, antioxidants and pH buffer.

Therefore, the present invention also relates to a liquid formulation (LF20′), which is the liquid formulation (LF20), wherein the amount of the at least one auxiliary agent is up to 40 wt-%, based on the total weight of the liquid formulation.

Particularly suitable auxiliary agents present in the liquid or solid formulation according to the present invention are selected from the group of glucose sirup, an additional antioxidant (other than Vitamin E), ascorbic acid, pH buffers, flavours and/or dyestuffs, preferably from the group of glucose sirup and/or ascorbic acid.

Suitable antioxidants to be used in the formulations according to the present invention are sulfites such as (SO₂ (E 220); Na₂SO₃ (E 221), NaHSO₃ (E 222); Na₂S₂O₅ (E 223); (K₂S₂O₅ (E 224); CaSO₃ (E 226); Ca(HSO₃)₂ (E 227); KHSO₃ (E 228)) and ascorbic acid. Most preferably in all embodiments of the present invention.

Preferably, in all embodiments of the present invention the liquid formulation comprises glucose sirup, more preferably in an amount selected in the range from 2 to 15 wt.-%, most preferably from 3 to 10 wt.-%, based on the total weight of the liquid formulation.

If the liquid formulation comprises ascorbic acid, the amounts are preferably selected in the range of 0.5 to 5 wt.-%, more preferably in the range from 0.75 to 3 wt.-%, most preferably in the range from 1 to 2 wt.-%, based on the total weight of the liquid formulation.

The liquid formulation according to the present invention can be produced using commonly known processes.

Generally, the liquid formulation (emulsion/dispersion) according to the present invention is produced as follows
(i) preparation of the lipid phase,
(ii) preparation of the aqueous phase,
and dispersion/emulsification of the lipid phase into the aqueous phase.

Thus, in a preferred embodiment the invention is also related to a process for the preparation of a liquid formulation according to the present invention, said process comprising
(i) preparation of a lipid phase by solubilizing the at least one cannabinoid in the at least one MCT oil, preferably at temperatures of 60 to 100° C., more preferably of 65 to 90° C., most preferably of 70 to 80° C., optionally followed by the addition of Vitamin E TGPS (if present),
(ii) preparation of an aqueous phase by solubilising the at least one emulsifier (other than Vitamin E TGPS) an, if present, the auxiliary agents in the water, followed by
(iii) emulsification of the lipid phase into the aqueous phase, preferably at temperatures of 60 to 100° C., more preferably of 65 to 90° C., most preferably of 70 to 85° C. to form an emulsion.

The preparation of the aqueous phase is suitably carried out at elevated temperatures according to well-known methods in the art. Before dispersion/emulsification it is further preferred that the aqueous phase is heated up to have approximately the same temperature as the lipid phase.

It is also possible to convert the liquid formulation according to the present invention into a solid (dried) formulation (which can be a powder, a beadlet, a granulate, etc).

This can be done by any commonly known and used technologies such as spray drying, spray granulating, etc., Preferably, the solid formulations according to the present invention is a spray dried solid formulation or in the form of a beadlet, which beadlets can be by spraying the emulsion into a fluidized bed of solid particles such as in particular of corn starch, which technique is e.g. exemplified in EP0285682 A1. It is well known to a person skilled in the art, that the drying may take place in the presence of a carrier, which may be added to the liquid formulation prior to drying or is used in the drying the process by spraying the liquid formulation into a carrier material.

Particular suitable carrier materials to be used in the context of the present invention encompass maltodextrin (which also may be used as thickener, i.e. having a dual role), corn starch and silica.

Thus, the present invention also relates to a process for the preparation of a solid preparation according to the present invention, said process comprising
(i) preparation of a lipid phase by solubilizing the at least one cannabinoid in the The at least one MCT oil, preferably at temperatures of 60 to 100° C., more preferably of 65 to 90° C., most preferably of 70 to 80° C., optionally followed by the addition of Vitamin E TGPS (if present),
(ii) preparation of an aqueous phase by solubilising the at least one emulsifier (other than Vitamin E TGPS) and, if present, the auxiliary agents in the water, followed by
(iii) emulsification of the lipid phase into the aqueous phase, preferably at temperatures of 60 to 100° C., more preferably of 65 to 90° C., most preferably of 70 to 80° C. to form an emulsion followed by
(iv) drying the emulsion.

The solid formulations according to the present invention are preferably prepared by spray drying according to standard methods in the art, preferably by using maltodextrin as carrier (auxiliary/flow) agent. Thus, step (iv) of the process preferably encompasses the step of drying the emulsion using maltodextrin as a carrier, even more preferably by spray drying. Preferably the maltodextrin is used in amounts of 2.5 to 15 wt.-%, more preferably in amounts of 3 to 10 wt.-%, most preferably in amounts of 5 to 10 wt.-%, based on the total weight of the solid formulation.

It is well understood that all preferences and definitions as given herein with regard e.g. to the concentration ranges also apply to the processes according to the present invention.

The conversion of the liquid formulation according to the present invention into a solid (dried) formulation means that the amount of the water is reduced to an amount under 5 wt-%, based on the total weight of the solid formulation.

Preferably, in a solid formulation (such as the ones made from the liquid formulation according to the present invention), the water content is below 3 wt-%, based on the total weight of the solid formulation.

More preferably, in a solid formulation (such as the ones made from the liquid formulation according to), the water content is 2.5 wt-% or lower, based on the total weight of the solid formulation.

Even more preferably, in a solid formulation (such as the ones made from the liquid formulation according to), the water content is below 2 wt-%, based on the total weight of the solid formulation.

It is well understood, that the amounts (wt.-%) of the ingredients (i), (ii) and (iii) as well as of the at least one optional auxiliary agent are shifting accordingly when the water is removed.

The definitions and preferences as provided herein for all ingredients are also applicable for the solid formulations as well.

Therefore, the present invention also relates to a solid formulation, which is the any of formulation (LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16), (LF16′), (LF17), (LF17′), (LF18), (LF19), (LF19′), (LF20) or (LF20′), wherein the water content is reduced to 0 to 5 wt-%, based on the total weight of the solid formulation.

Therefore, the present invention also relates to a solid formulation (SF) comprising
(i) 5-85 wt-%, based on the total weight of the solid formulation, of at least one cannabinoid, and
(ii) 2 to 25 wt-%, based on the total weight of the solid formulation, of the at least one MCT oil, and
(iii) 1 to 55 wt-%, based on the total weight of the solid formulation, of at least one emulsifier, and
(iv) optionally up to 40 wt-%, based on the total weight of the solid formulation, of at least one auxiliary agent, and
(v) less than 5 wt-%, based on the total weight of the solid formulation, of water,
wherein at least 50 wt-%, based on the total of the cannabinoid, of the at least one cannabinoid is in a non-crystalline form and
the ratio of the at least one cannabinoid to the at least one MCT oil is 2:1 to 4:1.

Therefore, the present invention also relates to a solid formulation (SF′) comprising
(i) 10-75 wt-%, based on the total weight of the solid formulation, of at least one cannabinoid, and
(ii) 5 to 20 wt-%, based on the total weight of the solid formulation, of the at least one MCT oil, and
(iii) 10 to 50 wt-%, based on the total weight of the solid formulation, of at least one emulsifier, and
(iv) optionally up to 40 wt-%, based on the total weight of the solid formulation, of at least one auxiliary agent, and
(v) less than 5 wt-%, based on the total weight of the solid formulation, of water,
wherein at least 50 wt-%, based on the total of the cannabinoid, of the at least one cannabinoid is in a non-crystalline form and
the ratio of the at least one cannabinoid to the at least one MCT oil is 2:1 to 4:1.

Therefore, the present invention also relates to a solid formulation (SF″), which it the solid formulation (SF), wherein the water content is below 3 wt-%, based on the total weight of the solid formulation.

Therefore, the present invention also relates to a solid formulation (SF′″), which it the solid formulation (SF), wherein the water content is equal or 2.5 wt.-%, even more preferably below 2 wt-%, based on the total weight of the solid formulation.

Therefore, the present invention also relates to a solid formulation (SF1), which is the solid formulation (SF), (SF′), (SF″) or (SF″), wherein at least one cannabinoid is chosen from the group consisting of cannabidiol, tetrahydrocannabinol, cannabinol, cannabigerol, cannabichromene, cannabicitran, cannabinodivarin, cannabiripsol, hexahydrocannabinol, delta-9-tetrahydrocannabiphoral, tetrahydrocannabivarin, endocannabinoid and endocannabinoid.

Therefore, the present invention also relates to a solid formulation (SF1′), which is the solid formulation (SF), (SF′), (SF″) or (SF″), wherein at least one cannabinoid is chosen from the group consisting of cannabidiol, tetrahydrocannabinol, cannabinol, cannabigerol, cannabichromene, cannabicitran, cannabinodivarin, cannabiripsol, hexahydrocannabinol and delta-9-tetrahydrocannabiphoral.

Therefore, the present invention also relates to a solid formulation (SF1″), which is the solid formulation (SF), (SF′), (SF″) or (SF″), wherein at least one cannabinoid is chosen from the group consisting of cannabidiol, tetrahydrocannabinol, cannabinol and cannabigerol (most preferred is cannabidiol).

Therefore, the present invention also relates to a solid formulation (SF2), which is the solid formulation (SF), (SF′), (SF″), (SF″), (SF1), (SF1′) or (SF1″), wherein the amount of the of the at least one cannabinoid in the formulation according to the present invention is 15-75 wt-%, based on the total weight of the solid formulation.

Therefore, the present invention also relates to a solid formulation (SF2′), which is the solid formulation (SF), (SF′), (SF″), (SF″), (SF1), (SF1′) or (SF1″), wherein the amount of the of the at least one cannabinoid in the formulation according to the present invention is 15-70 wt-%, based on the total weight of the solid formulation.

Therefore, the present invention also relates to a solid formulation (SF2″), which is the solid formulation (SF), (SF′), (SF″), (SF″), (SF1), (SF1′) or (SF1″), wherein the amount of the of the at least one cannabinoid in the formulation according to the present invention is 20-65 wt-%, based on the total weight of the solid formulation.

Therefore, the present invention also relates to a solid formulation (SF3), which is the solid formulation (SF), (SF′), (SF″), (SF″), (SF1), (SF1′), (SF1″), (SF2), (SF2′) or (SF2″), wherein at least 55 wt-%, based on the total weight of the cannabinoid, of the one cannabinoid is in a non-crystalline form.

Therefore, the present invention also relates to a solid formulation (SF4), which is the solid formulation (SF), (SF′), (SF″), (SF″), (SF1), (SF1′), (SF1″), (SF2), (SF2′) or (SF2″), wherein at least 60 wt-%, based on the total weight of the cannabinoid, of the one cannabinoid is in a non-crystalline form.

Therefore, the present invention also relates to a solid formulation (SF5), which is the solid formulation (SF), (SF′), (SF″), (SF″), (SF1), (SF1′), (SF1″), (SF2), (SF2′) or (SF2″), wherein at least 65 wt-%, based on the total weight of the cannabinoid, of the one cannabinoid is in a non-crystalline form.

Therefore, the present invention also relates to a solid formulation (SF6), which is the solid formulation (SF), (SF′), (SF″), (SF″), (SF1), (SF1′), (SF1″), (SF2), (SF2′) or (SF2″), wherein at least 70 wt-%, based on the total weight of the cannabinoid, of the one cannabinoid is in a non-crystalline form.

Therefore, the present invention also relates to a solid formulation (SF7), which is the solid formulation (SF), (SF′), (SF″), (SF″), (SF1), (SF1′), (SF1″), (SF2), (SF2′) or (SF2″), wherein at least 75 wt-%, based on the total weight of the cannabinoid, of the one cannabinoid is in a non-crystalline form.

Therefore, the present invention also relates to a solid formulation (SF8), which is the solid formulation (SF), (SF′), (SF″), (SF″), (SF1), (SF1′), (SF1″), (SF2), (SF2′) or (SF2″), wherein at least 80 wt-%, based on the total of the cannabinoid, of the one cannabinoid is in a non-crystalline form.

Therefore, the present invention also relates to a solid formulation (SF9), which is the solid formulation (SF), (SF′), (SF″), (SF″), (SF1), (SF1′), (SF1″), (SF2), (SF2′) or (SF2″), wherein at least 85 wt-%, based on the total weight of the cannabinoid, of the one cannabinoid is in a non-crystalline form.

Therefore, the present invention also relates to a solid formulation (SF10), which is the solid formulation (SF), (SF′), (SF″), (SF″), (SF1), (SF1′), (SF1″), (SF2), (SF2′) or (SF2″), wherein at least 90 wt-%, based on the total weight of the cannabinoid, of the one cannabinoid is in a non-crystalline form.

Therefore, the present invention also relates to a solid formulation (SF11), which is the solid formulation (SF), (SF′), (SF″), (SF″), (SF1), (SF1′), (SF1″), (SF2), (SF2′) or (SF2″), wherein at least 95 wt-%, based on the total weight of the cannabinoid, of the one cannabinoid is in a non-crystalline form.

Therefore, the present invention also relates to a solid formulation (SF12), which is the solid formulation (SF), (SF′), (SF″), (SF″), (SF1), (SF1′), (SF1″), (SF2), (SF2′) or (SF2″), wherein at least 98 wt-%, based on the total weight of the cannabinoid, of the one cannabinoid is in a non-crystalline form.

Therefore, the present invention also relates to a solid formulation (SF13), which is the solid formulation (SF), (SF′), (SF″), (SF″), (SF1), (SF1′), (SF1″), (SF2), (SF2′) or (SF2″), wherein 100 wt-%, based on the total weight of the cannabinoid, of the one cannabinoid is in a non-crystalline form.

Therefore, the present invention also relates to a solid formulation (SF14), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12) or (SF13), wherein the at least one MCT oil is chosen from the group consisting of caproic acid, caprylic acid, capric acid and lauric acid.

Therefore, the present invention also relates to a solid formulation (SF15), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13) or (SF14), wherein the content of the at least one MCT oil is 8 to 18 wt-%, based on the total weight of the solid formulation.

Therefore, the present invention also relates to a solid formulation (SF16), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15) or (SF15′), wherein the ratio of the at least one cannabinoid to the at least one MCT oil is 2:1 to 3.5:1.

Therefore, the present invention also relates to a solid formulation (SF16′), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15) or (SF15′), wherein the ratio of the at least one cannabinoid to the at least one MCT oil is 2.2:1 to 3.5:1.

Therefore, the present invention also relates to a solid formulation (SF17), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15), (SF15′), (SF16) or (SF16′), wherein the at least one emulsifier is chosen from the group consisting of modified (food) starches, vitamin E TPGS (CAS no 9002-96-4), ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia (=gum arabic), modified gum acacia, TIC gum, flaxseed gum, ghatti gum, tamarind gum and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof).

Therefore, the present invention also relates to a solid formulation (SF17′), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15), (SF15′), (SF16) or (SF16′), wherein the at least one emulsifier is chosen from the group consisting of modified (food) starches, vitamin E TPGS, polysaccharide gums, gelatine (bovine, fish, pork, poultry) and plant proteins.

Therefore, the present invention also relates to a solid formulation (SF18), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15), (SF15′), (SF16), (SF16′), (SF17) or (SF17′), wherein the amount of the at least one emulsifier is 15 to 50 wt-%, based on the total weight of the solid formulation.

Therefore, the present invention also relates to a solid formulation (SF19), which is the solid formulation (SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15), (SF15′), (SF16), (SF16′), (SF17), (SF17′) or (SF18), wherein the solid formulation comprises at least one auxiliary agent chosen from the group consisting of dyestuffs, thickeners (such as maltodextrin, glucose syrup), fillers, binders, flavours, antioxidants and pH buffer.

Therefore, the present invention also relates to a solid formulation (SF19′), which is the solid formulation (SF19), wherein the amount of the at least one auxiliary agent is up to 35 wt-%, based on the total weight of the solid formulation.

Particularly suitable auxiliary agents present in the solid formulation according to the present invention are selected from the group of (dried) glucose sirup, maltodextrin, ascorbic acid, pH buffers, flavours and/or dyestuffs, preferably from the group of (dried) glucose sirup, maltodextrin and/or ascorbic acid.

In all embodiments of the present invention, it is particularly preferred that the liquid and/or the solid formulation comprises as emulsifier Vitamin E TPGS and at least one other emulsifier (other than vitamin E TPGS) such as in particular gelatine.

In all embodiments of the present invention the use of gelatine having a bloom value of less than 200 such as in the range of 100 to 200 is particularly preferred.

In all embodiments of the present invention, it is particularly preferred if the liquid and solid formulations according to the present invention contain no further oil next to the at least one MCT oil, i.e. the MCT oil is the sole oil, which is used in the preparation process to solubilize the at least one cannabinoid present in the formulations according to the present invention.

Thus, particularly preferred formulations according to the present invention are the following:
A liquid formulation (A) consisting essentially of
(i) 7.5 to 50 wt-%, preferably 10 to 40 wt.-%, most preferably 15-30 wt.-% based on the total weight of the liquid formulation, of at least one cannabinoid, and
(ii) 3 to 20 wt-%, preferably 3 to 15 wt.-%, most preferably 3 to 10 wt.-%, based on the total weight of the liquid formulation, of at least one MCT oil, and
(iii) 0 to 5 wt.-%, preferably 0 to 4 wt.-%, most preferably 0 to 3.5 wt.-% of Vitamin E TPGS,
(iv) 15 to 50 wt-%, preferably 20 to 40 wt.-%, most preferably 20 to 35 wt.-%, based on the total weight of the liquid formulation, of at least one emulsifier, and (v) 1 to 15 wt.-%, preferably 2.5 to 12 wt.-%, most preferably 4 to 10 wt.-% of glucose sirup, and
(vi) 0 to 5 wt.-%, preferably 0 to 4 wt.-%, most preferably 0 to 3 wt. % of at least one auxiliary agent chosen from the group of antioxidants (other than Vitamin E), pH buffers, flavour and/or dyestuffs, and
(vii) 12 to 55 wt-%, preferably 15 to 50 wt.-%, most preferably 15 to 45 wt.-%, based on the total weight of the liquid formulation, of water,
wherein at least 50 wt-%, based on the total weight of the cannabinoid, of the at least one cannabinoid is in a non-crystalline form, and
wherein the ratio of the at least one cannabinoid to the at least one MCT oil is 2:1 to 4:1.

It is well understood that all preferences and definitions as given herein also apply to the liquid formulation (A) such as in particular:
(a) the emulsifier is selected from gelatine or OSA starch, even more preferably the emulsifier is gelatine or a mixture of gelatine and Vitamin E TPGS, and/or
(b) the MCT oil is chosen from the group consisting of caproic acid, caprylic acid, capric acid and lauric acid, and/or
(c) the gelatine is a gelatine having a bloom value selected in the range of 100-200, and/or
(d) at least 75 wt.-% of the at least cannabinoid is in a non-crystalline form, and/or
(e) the at least one cannabinoid is CBD and/or
(f) the additional antioxidant (other than the Vitamin E) is ascorbic acid.
A liquid formulation (B) consisting essentially of
(i) 10 to 40 wt-%, preferably 15 to 35 wt.-%, most preferably 20-30 wt.-% based on the total weight of the liquid formulation, of at least one cannabinoid, and
(ii) 3 to 20 wt-%, preferably 4 to 15 wt.-%, most preferably 5 to 15 wt.-%, based on the total weight of the liquid formulation, of the at least one MCT oil, and
(iii) 0 to 5 wt.-%, preferably 0 to 4 wt.-%, most preferably 0 to 3.5 wt.-% of Vitamin E TPGS,
(iv) 15 to 50 wt-%, preferably 20 to 40 wt.-%, most preferably 20 to 35 wt.-%, based on the total weight of the liquid formulation, of gelatine, and
(v) 1 to 15 wt.-%, preferably 2.5 to 12 wt.-%, most preferably 4 to 10 wt.-% of glucose sirup, and
(vi) 0 to 5 wt.-%, preferably 0 to 4 wt.-%, most preferably 0 to 3 wt. % of at least one auxiliary agent chosen from the group of antioxidants (other than Vitamin E), pH buffers, flavour and/or dyestuffs, and
(vii) 12 to 45 wt-%, preferably 15 to 40 wt.-%, most preferably 15 to 35 wt.-%, based on the total weight of the liquid formulation, of water,
wherein at least 50 wt-%, based on the total weight of the cannabinoid, of the at least one cannabinoid is in a non-crystalline form, and wherein the ratio of the at least one cannabinoid to the at least one MCT oil is 2:1 to 4:1.

It is again well understood that all preferences and definitions as given herein also apply to the liquid formulation (B) such as in particular:
(a) the emulsifier is a mixture of gelatine and Vitamin E TPGS, and/or
(b) at least 75 wt.-%, preferably at least 85 wt.-%, most preferably at least 95 wt.-% such as at least 99 wt.-% of the at least cannabinoid is in a non-crystalline form, and/or
(c) the MCT oil is chosen from the group consisting of caproic acid, caprylic acid, capric acid and lauric acid, and/or
(d) the gelatine is a gelatine having a bloom value selected in the range of 100-200, and/or
(e) the at least one cannabinoid is CBD, and/or
(f) the additional antioxidant (other than the Vitamin E) is ascorbic acid.
A solid formulation (C) consisting essentially of
(i) 10 to 50 wt-%, preferably 20 to 45 wt.-%, most preferably 30-45 wt.-% based on the total weight of the solid formulation, of at least one cannabinoid, and
(ii) 3 to 20 wt-%, preferably 3 to 15 wt.-%, most preferably 5 to 15 wt.-%, based on the total weight of the solid formulation, of at least one MCT oil, and
(iii) 0 to 7.5 wt.-%, preferably 0 to 7 wt.-%, most preferably 0 to 5.5 wt.-%, based on the total weight of the solid formulation, of Vitamin E TPGS,
(iv) 15 to 50 wt-%, preferably 20 to 40 wt.-%, most preferably 20 to 35 wt.-%, based on the total weight of the solid formulation, of at least one emulsifier (other than the Vitamin E TPGS), and
(v) 1 to 20 wt.-%, preferably 5 to 20 wt.-%, most preferably 5 to 15 wt.-%, based on the total weight of the solid formulation, of glucose sirup, and
(vi) 0 to 5 wt.-%, preferably 0 to 4 wt.-%, most preferably 0 to 3 wt. %, based on the total weight of the solid formulation, of at least one auxiliary agent chosen from the group of antioxidants (other than Vitamin E), pH buffers, flavour and/or dyestuffs, and
(vii) 0 to 15 wt.-%, preferably 2.5 to 10 wt.-%, most preferably 5 to 10 wt.-% based on the total weight of the solid formulation, of a carrier, and
(viii) less than 5 wt-%, preferably 0 to 3.5 wt.-%, based on the total weight of the solid formulation, of water,
wherein at least 50 wt-%, based on the total weight of the cannabinoid, of the at least one cannabinoid is in a non-crystalline form, and
wherein the ratio of the at least one cannabinoid to the at least one MCT oil is 2:1 to 4:1.

It is well understood that all preferences and definitions as given herein also apply to the solid formulation (C) such as in particular:
(a) the emulsifier is selected from gelatine or OSA starch, even more preferably the emulsifier is gelatine or a mixture of gelatine and Vitamin E TPGS, and/or
(b) the MCT oil is chosen from the group consisting of caproic acid, caprylic acid, capric acid and lauric acid, and/or
(c) the gelatine is a gelatine having a bloom value selected in the range of 100-200, and/or
(d) the carrier is maltodextrin or silica, and/or
(e) at least 75 wt.-% of the at least cannabinoid is in a non-crystalline form, and/or
(f) the at least one cannabinoid is CBD, and/or
(g) the additional antioxidant (other than the Vitamin E) is ascorbic acid, and/or
(h) the solid formulation is a spray dried solid formulation.
A solid formulation (D) consisting essentially of
(i) 10 to 50 wt-%, preferably 20 to 45 wt.-%, most preferably 30-40 wt.-% based on the total weight of the solid formulation, of at least one cannabinoid, and
(ii) 3 to 20 wt-%, preferably 4 to 15 wt.-%, most preferably 5 to 15 wt.-%, based on the total weight of the solid formulation, of at least one MCT oil, and
(iii) 0 to 5 wt.-%, preferably 0 to 4 wt.-%, most preferably 0 to 3.5 wt.-%, based on the total weight of the solid formulation, of Vitamin E TPGS, and
(iv) 15 to 50 wt-%, preferably 20 to 40 wt.-%, most preferably 20 to 35 wt.-%, based on the total weight of the solid formulation, of gelatine, and
(v) 1 to 15 wt.-%, preferably 2.5 to 12 wt.-%, most preferably 4 to 10 wt.-%, based on the total weight of the solid formulation, of glucose sirup, and
(vi) 0 to 5 wt.-%, preferably 0 to 4 wt.-%, most preferably 0 to 3 wt. %, based on the total weight of the solid formulation, of at least one auxiliary agent chosen from the group of of antioxidants (other than Vitamin E), pH buffers, flavour and/or dyestuffs, and
(vii) 0 to 15 wt.-%, preferably 3 to 10 wt.-%, most preferably 5 to 10 wt.-% based on the total weight of the solid formulation, of a carrier, and
(viii) less than 5 wt-%, preferably 0 to 3.5 wt.-%, based on the total weight of the solid formulation, of water.
wherein at least 50 wt-%, based on the total weight of the cannabinoid, of the at least one cannabinoid is in a non-crystalline form, and
wherein the ratio of the at least one cannabinoid to the at least one MCT oil is 2:1 to 4:1.

It is again well understood that all preferences and definitions as given herein also apply to the solid formulation (D) such as in particular:
(a) the emulsifier is a mixture of gelatine and Vitamin E TPGS, and/or
(b) at least 75 wt.-%, preferably at least 85 wt.-%, most preferably at least 95 wt.-% such as at least 99 wt.-% of the at least cannabinoid is in a non-crystalline form, and/or
(c) the MCT oil is chosen from the group consisting of caproic acid, caprylic acid, capric acid and lauric acid, and/or
(d) the gelatine is a gelatine having a bloom value in the range of 100-200, and/or
(e) the carrier is maltodextrin or silica, and/or
(f) the at least one cannabinoid is CBD, and/or
(g) the additional antioxidant (other than the Vitamin E) is ascorbic acid and/or
(h) the solid formulation is a spray dried solid formulation.

Even more advantageous in all embodiments of the present invention is a solid formulation (E) consisting essentially of
(i) 30-45 wt.-% based on the total weight of the solid formulation, of CBD, and
(ii) 5 to 15 wt.-%, based on the total weight of the solid formulation, of at least one MCT oil, and
(iii) 0 to 5.5 wt.-%, based on the total weight of the solid formulation, of Vitamin E TPGS, and
(iv) 20 to 35 wt.-%, based on the total weight of the solid formulation, of gelatine, and
(v) 4 to 10 wt.-%, based on the total weight of the solid formulation, of glucose sirup, and
(vi) 0 to 3 wt. %, based on the total weight of the solid formulation, of an additional antioxidant (other than Vitamin E), preferably ascorbic acid, and
(vii) 0 to 10 wt.-% based on the total weight of the solid formulation, of maltodextrin,
(viii) less than 5 wt.-%, based on the total weight of the solid formulation, of water.

Most preferred in all embodiments of the present invention is a solid formulation (F) consisting essentially of
(i) 35-42 wt.-% based on the total weight of the solid formulation, of CBD, and
(ii) 5 to 15 wt.-%, based on the total weight of the solid formulation, of at least one MCT oil, and
(iii) 2.5 to 5.5 wt.-%, based on the total weight of the solid formulation, of Vitamin E TPGS, and
(iv) 25 to 35 wt.-%, based on the total weight of the solid formulation, of gelatine, and
(v) 4 to 10 wt.-%, based on the total weight of the solid formulation, of glucose sirup, and
(vi) 1 to 3 wt. %, based on the total weight of the solid formulation, based on the total weight of the solid formulation, of an additional antioxidant (other than Vitamin E), preferably ascorbic acid, and
(vii) 0 to 10 wt.-% based on the total weight of the solid formulation, of maltodextrin,
(viii) less than 5 wt.-%, based on the total weight of the solid formulation, of water.

Another advantageous solid formulation according to the present invention is a solid formulation (G), consisting essentially of
(i) 35-42 wt.-% based on the total weight of the solid formulation, of CBD, and
(ii) 5 to 15 wt.-%, based on the total weight of the solid formulation, of at least one MCT oil, and
(iii) 2.5 to 5.5 wt.-%, based on the total weight of the solid formulation, of Vitamin E TPGS, and
(iv) 25 to 35 wt.-%, based on the total weight of the solid formulation, of gelatine, and
(v) 4 to 10 wt.-%, based on the total weight of the solid formulation, of glucose sirup, and
(vi) 1 to 3 wt. %, based on the total weight of the solid formulation, of an additional antioxidant (other than Vitamin E), preferably ascorbic acid, and
(vii) 0 to 10 wt.-% based on the total weight of the solid formulation, of maltodextrin,
(viii) less than 5 wt.-%, based on the total weight of the solid formulation, of water.

The term ‘consisting essentially of’ as used in the context of the invention means that the addition of the wt-% of the listed ingredients add up to 100 wt.-%. However, it cannot be excluded that small amounts of impurities may be present such as e.g. in amounts of less than 5 wt.-%, preferably less than 3 wt.-% which are introduced via the respective raw materials or processes used.

The term ‘less than 5 wt.-% of water’ refers to any amount selected in the range from 0 to <5 wt.-%, such as from 0 to 4.999 wt.-%, based on the total weight of the solid formulation. Particular advantageous formulation according to the present invention comprise from 1 to <5 wt.-%, from 2 to <5 wt.-% or from 2.5 to <5 wt.-% of water, based on the total weight of the solid formulation.

The solid formulations according to the present invention are preferably in the form of particles having a preferred particle size of less than 1.5 mm, even more preferred of less than 1 mm. Said particle size of the solid formulations can e.g. be determined by sieving according to standard methods in the art.

In another preferred embodiment the solid formulations according to the present invention are incorporated into dosage forms suitable for oral application such as a tablet (orally dispersable tablet, chewable tablet, film coated tablet, immediate release tablet, extended or sustained release tablet and similar), a capsule, an orally dispersable film, or a gummy.

The amount of the solid formulation of the present invention to be incorporated into such an oral dosage form is preferably selected in the range from 10 mg to 500 mg.

To prepare a solid dosage form according to the present invention, the solid formulations according to the present invention may be admixed with excipients known in the art such as
diluents, like lactose, starch, microcrystalline cellulose, sorbitol, mannitol, dibasic calcium phosphate dihydrate, calcium sulfate dihydrate, sucrose-based diluents and mixtures thereof;
binders, like acacia, cellulose derivatives, gelatin, glucose, polyvinylpyrollidone, starch, sucrose, sorbitol, tragacanth, sodium alginate and mixtures thereof;
disintegrants, like microcrystalline cellulose and cellulose derivatives, starch and its derivatives, alginic acid and its derivatives, ion-exchange resins, cross-linked sodium carboxymethyl cellulose, sodium starch glycolate, cross-linked polyvinylpyrrolidone and formaldehyde-caseine;
lubricants, antiadherents and glidants, like magnesium-, calcium- and sodium stearates, stearic acid, hydrogenated castor oil, talc, water, polyethylene glycol, sodium lauryl sulfate, magnesium lauryl sulfate and silica.

The liquid formulation according to the present invention such as the liquid formulations [(LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16), (LF16′), (LF17), (LF17′), (LF18), (LF19), (LF19′), (LF20) or (LF20′)] as well as the solid formulation [(SF), (SF′), (SF″), (SF′″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15), (SF15′), (SF16), (SF16′), (SF17), (SF17′), (SF18), (SF19) or (SF19′)] according to the present invention can be used in a variety of fields of application, such as food, feed, dietary supplements, pharmaceuticals, and personal care products.

These products can be in any form (such as solid, liquid or gel-like).

Preferred are food, dietary supplements, pharmaceuticals, and personal care products comprising at least one liquid formulation according to the present invention such as the liquid formulations [(LF), (LF1), (LF1′), (LF1″), (LF2), (LF2′), (LF2″), (LF3), (LF4), (LF5), (LF6), (LF7), (LF8), (LF9), (LF10), (LF11), (LF12), (LF13), (LF14), (LF15), (LF16), (LF16′), (LF17), (LF17′), (LF18), (LF19), (LF19′), (LF20) or (LF20′)] and/or at least one solid formulation [(SF), (SF′), (SF″), (SF″), (SF1), (SF1′), (SF1″), (SF2), (SF2′), (SF2″), (SF3), (SF4), (SF5), (SF6), (SF7), (SF8), (SF9), (SF10), (SF11), (SF12), (SF13), (SF14), (SF15), (SF15′), (SF16), (SF16′), (SF17), (SF17′), (SF18), (SF19) or (SF19′)] according to the present invention.

The invention is also concerned with the following embodiments:
1. A solid formulation comprising
   (i) 10 to 50 wt-%, based on the total weight of the solid formulation, of at least one cannabinoid, and
   (ii) 3 to 20 wt-%, based on the total weight of the solid formulation, of Vitamin E, and (iii) 0 to 7.5 wt.-%, based on the total weight of the solid formulation, of Vitamin E TPGS, and
   (iv) 15 to 50 wt-%, based on the total weight of the solid formulation, of at least one emulsifier (other than the Vitamin E TPGS), and
   (v) 1 to 40 wt.-%, based on the total weight of the solid formulation, of at least one auxiliary agent, and
   (vi) less than 5 wt-%, based on the total weight of the solid formulation, of water,
      wherein at least 50 wt-%, based on the total weight of the cannabinoid, of the at least one cannabinoid is in a non-crystalline form, and
      wherein the ratio of the at least one cannabinoid to Vitamin E is 2:1 to 4:1.
2. The solid formulation according to embodiment 1, comprising
   (i) 10 to 50 wt-%, based on the total weight of the solid formulation, of at least one cannabinoid, and
   (ii) 3 to 20 wt-%, based on the total weight of the solid formulation, of at least one MCT oil, and
   (iii) 0 to 7.5 wt.-%, based on the total weight of the solid formulation, of Vitamin E TPGS, and
   (iv) 15 to 50 wt-%, based on the total weight of the solid formulation, of at least one emulsifier (other than the Vitamin E TPGS), and
   (v) 1 to 40 wt.-%, based on the total weight of the solid formulation, of at least one auxiliary agent, and
   (vi) less than 5 wt-%, based on the total weight of the solid formulation, of water, wherein at least 50 wt-%, based on the total weight of the cannabinoid, of the at least one cannabinoid is in a non-crystalline form, and wherein the ratio of the at least one cannabinoid to the at least one MCT oil is 2:1 to 4:1.
3. The solid formulation according to embodiment 2, wherein the formulation consists essentially of the ingredients (i) to (vi).
4. The solid formulation according to anyone of embodiment v1 to 3, wherein the amount of the at least one cannabinoid is selected in the range from 20 to 45 wt.-%, preferably in the range from 30 to 45 wt.-%, most preferably in the range of 35 to 40 wt.-%, based on the total weight of the solid formulation.
5. The solid formulation according to anyone of embodiment 1 to 4, wherein the amount of the at least one MCT oil is selected in the range from 3 to 15 wt.-%, preferably in the range from 5 to 15 wt.-%, based on the total weight of the solid formulation.
6. The solid formulation according to anyone of embodiments 1 to 5, wherein the amount of Vitamin E TPGS is selected in the range from 0 to 7 wt.-%, preferably in the range from 0 to 5.5 wt.-%, even more preferably in the range from 2 to 5.5 wt.-%, based on the total weight of the solid formulation.
7. The solid formulation according to anyone of embodiments 1 to 6, wherein the amount of the at least emulsifier is selected in the range from 20 to 40 wt.-%, preferably in the range from 20 to 35 wt.-%, based on the total weight of the solid formulation.
8. The solid formulation according to anyone of embodiments 1 to 7, wherein the amount of the at least one auxiliary agent is selected in the range from 5 to 34 wt.-%, preferably in the range from 5 to 28 wt.-%, based on the total weight of the solid formulation.
9. The solid formulation according to anyone of embodiments 1 to 8, wherein the amount of water is selected in the range from 0 to less than 5 wt.-%, based on the total weight of the solid formulation.
10. The solid formulation according to anyone of embodiments 1 to 9, wherein the at least one cannabinoid is selected from the group of cannabidiol, tetrahydrocannabinol, cannabinol, cannabigerol, cannabichromene, cannabicitran, cannabinodivarin, cannabiripsol, hexahydrocannabinol, delta-9-tetrahydrocannabiphoral, tetrahydrocannabivarin, endocannabinoid and endocannabinoid.
11. The solid formulation according to anyone of embodiments 1 to 10, wherein the at least one cannabinoid is cannabidiol.
12. The solid formulation according to anyone of embodiments 1 to 11, wherein the at least one MCT oil is chosen from the group consisting of caproic acid, caprylic acid, capric acid and lauric acid.
13. The solid formulation according to anyone of embodiments 1 to 12, wherein the emulsifier is gelatine (bovine, fish, pork, poultry, preferably pork) or modified (OSA) starch.
14. The solid formulation according to embodiment 13, wherein the gelatine has a bloom value of less than 200, preferably selected in the range from 100 to 200,
15. The solid formulation according to anyone of embodiments 1 to 14, wherein the at least one auxiliary agent is selected from the group of dyestuffs, thickeners, carriers, fillers, binders, flavours, antioxidants (other than Vitamin E) and pH buffers.
16. The solid formulation according to anyone of embodiments 1 to 15, wherein the at least one auxiliary agent is selected from the group of thickeners, carriers and antioxidants (other than Vitamin E).
17. The solid formulation according to claim 16, wherein the thickener is selected from the group of (dried) glucose sirup, maltodextrin as well as mixtures thereof.
18. The solid formulation according to claim 16 or 17, wherein the carrier is selected from the group of maltodextrin, corn starch and/or silica.
19. The solid formulation according to embodiment 17, 18 or 19, wherein the additional antioxidant is selected from the group of sulfites and/or ascorbic acid, preferably the additional antioxidant is ascorbic acid.
20. The solid formulation according to anyone of embodiments 1 to 19, wherein the at least one auxiliary agent encompasses at least one thickener and at least one antioxidant (other than Vitamin E), even more preferably at least one thickener, at least one carrier and at least one antioxidant (other than Vitamin E).
21. The solid formulation according to anyone of embodiments 17 to 20, wherein the amount of glucose sirup in the solid formulation is selected in the range from 1 to 20 wt.-%, preferably 5 to 20 wt.-%, most preferably 5 to 15 wt.-%, based on the total weight of the solid formulation.
22. The solid formulation according to anyone of embodiments 17 to 21, wherein the amount of maltodextrin in the solid formulation is selected in the range of 2.5 to 15 wt.-%, preferably 3 to 10 wt.-%, most preferably 5 to 10 wt.-%, based on the total weight of the solid formulation.
23. The solid formulation according to anyone of embodiments 17 to 22, wherein the amount of the additional antioxidant, preferably of ascorbic acid in the solid formulation is selected in the range from 1 to 3 wt.-%, based on the total weight of the solid formulation.
24. The solid formulation according to anyone of embodiments 1 to 23, wherein the solid formulation is a spray dried solid formulation.
25. The solid formulation according to anyone of the embodiments 1 to 24, wherein at least 75 wt.-% of the at least cannabinoid is in a non-crystalline form.
26. Use of a solid formulation according to anyone of embodiments 1 to 25 in food products, feed products, dietary supplements, pharmaceutical compositions, and personal care products.
27. Use of a formulation according to any of the embodiments 1 to 25 in pharmaceutical products.
28. A food product, feed product, dietary supplement, pharmaceutical, and personal care product comprising a formulation according to any of the embodiments 1 to 25.
29. The pharmaceutical product according to claim 28, wherein the pharmaceutical product is in the form of a tablet, a capsule, an orally dispersable film, or a gummy.
30. Use of MCT to enhance the bioavailability of at least one cannabinoid, preferably of cannabidiol, most preferably in the form of a solid formulation according to anyone of the embodiments 1 to 25.
31. Use of at least one MCT oil to reduce or inhibit the formation of crystals of at least one cannabinoid, preferably of cannabidiol, most preferably in the form of a solid formulation such as the solid formulations according to anyone of the embodiments 1 to 25.
32. A process for the preparation of a solid formulation according to anyone of embodiments 1 to 25, said process encompassing the steps of
   (i) solubilising the at least one cannabinoid in the at least one MCT oil, optionally followed by the addition of Vitamin E TGPS to form a lipid phase,
   (ii) solubilising the at least one emulsifier (other than Vitamin E TGPS) and optionally one or more of the auxiliary agents in the water to form an aqueous phase, followed by
   (iii) emulsification of the lipid phase into the aqueous phase, followed by
   (iv) drying the emulsion.
33. The process according to embodiment 32, wherein step (i) is carried out at a temperature selected in the range from 60 to 100° C., preferably from 65 to 90° C., most preferably from 70 to 80° C.
34. The process according to embodiment 32 or 33, wherein the emulsification is carried out at a temperature selected in the range from 60 to 100° C., more preferably of 65 to 90° C., most preferably of 70 to 80° C.
35. The process according to anyone of embodiments 32 to 34, wherein the drying is spray drying, preferably using maltodextrin as auxiliary (carrier) agent.
36. The solid formulation according to anyone of the preceding claims, wherein the degree of crystallinity (in %) is determined by DSC using the respective crystalline cannabinoid as external standard.

It is well understood that all definitions and preferences as outlined herein also apply to the embodiments 1 to 33.

Further preferred embodiments are as follows
1a. A liquid formulation comprising
   (i) 5-85 wt-%, based on the total weight of the liquid formulation, of at least one cannabinoid, and
   (ii) 2 to 25 wt-%, based on the total weight of the liquid formulation, of MCT, and (iii) 1 to 50 wt-%, based on the total weight of the liquid formulation, of at least one emulsifier, and
   (iv) 12 to 55 wt-%, based on the total weight of the liquid formulation, of water,
   wherein at least 50 wt-%, based on the total weight of the cannabinoid, of the at least one cannabinoid is in a non-crystalline form and
   the ratio of the at least one cannabinoid to MCT is 2:1 to 4:1.
2a. Liquid formulation according to embodiment 1a, wherein at least one cannabinoid is chosen from the group consisting of cannabidiol, tetrahydrocannabinol, cannabinol, cannabigerol, cannabichromene, cannabicitran, cannabinodivarin, cannabiripsol, hexahydrocannabinol, delta-9-tetrahydrocannabiphoral, tetrahydrocannabivarin, endocannabinoid and endocannabinoid.
3a. Liquid formulation according to embodiment 1a or embodiment 2a, wherein the amount of the of the at least one cannabinoid in the formulation according to the present invention is 10-85 weight-%, based on the total weight of the liquid formulation.
4a. Liquid formulation according to any of the preceding embodiments (1a to 3a), wherein at least 55 wt-%, based on the total weight of the cannabinoid, of the one cannabinoid is in a non-crystalline form.
5a. Liquid formulation according to any of the preceding embodiments (1a to 4a), wherein the MCT oil is chosen from the group consisting of caproic acid, caprylic acid, capric acid and lauric acid.
6a. Liquid formulation according to any of the preceding embodiments (1a to 5a), wherein the content of MCT oil is 3 to 20 wt-%, based on the total weight of the liquid formulation.
7a. Liquid formulation according to any of the preceding embodiments (1a to 6a), wherein the at least one emulsifier is chosen from the group consisting of modified (food) starches, Vitamin E TPGS, ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia (=gum arabic), modified gum acacia, TIC gum, flaxseed gum, ghatti gum, tamarind gum and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof).
8a. Liquid formulation according to any of the preceding embodiments (1a-7a), wherein the liquid formulation comprises at least one auxiliary agent chosen from the group consisting of dyestuffs, thickeners, fillers, binders, flavours, antioxidants and pH buffer.
9a. Liquid formulation according to embodiment 8a, wherein the amount of the at least one auxiliary agent is up to 40 wt-%, based on the total weight of the liquid formulation.
10a. A solid formulation comprising
   (i) 5-85 wt-%, based on the total weight of the solid formulation, of at least one cannabinoid, and
   (ii) 2 to 25 wt-%, based on the total weight of the solid formulation, of MCT oil, and
   (iii) 1 to 50 wt-%, based on the total weight of the solid formulation, of at least one emulsifier, and
   (iv) optionally up to 40 wt-%, based on the total weight of the solid formulation, of at least one auxiliary agent, and
   (v) less than 5 wt-%, based on the total weight of the solid formulation, of water,
   wherein at least 50 wt-%, based on the total weight of the cannabinoid, of the at least one cannabinoid is in a non-crystalline form and
   the ratio of the at least one cannabinoid to MCT oil is 2:1 to 4:1.
11a. Solid formulation according to embodiment 10a, wherein the water content is below 3 wt-%, based on the total weight of the solid formulation.
12a. Solid formulation according to embodiment 10a or embodiment 11a, wherein at least one cannabinoid is chosen from the group consisting of cannabidiol, tetrahydrocannabinol, cannabinol, cannabigerol, cannabichromene, cannabicitran, cannabinodivarin, cannabiripsol, hexahydrocannabinol and delta-9-tetrahydrocannabiphoral.
13a. Use of a formulation according to any of the preceding embodiments in food products, feed products, dietary supplements, pharmaceutical compositions, and personal care products.
14a. Use of a formulation according to any of the preceding embodiments 1a-12a in pharmaceutical compositions.
15a. A food product, feed product, dietary supplement, pharmaceutical, and personal care product comprising a formulation according to any of the preceding embodiment 1a-12a.
16a. A pharmaceutical composition comprising a formulation according to any of the preceding embodiments 1a-12a.

The following examples are provided to further illustrate the compositions and effects of the present invention. These examples are illustrative only and are not intended to limit the scope of the invention in any way.

All the parts and percentages in the Examples are related to the weight (when not otherwise stated) and the temperature is given in° C. (when not otherwise stated).

### EXAMPLES

### General Methods

The degree of crystallinity of the solid formulations was measured using Differential Scanning calorimetry (DSC) as outlined in the following:

External standard: Crystalline CBD (1-2 mg) was placed in an aluminum pan which was then hermetically sealed (external standard). An empty pan was used as reference. The respective samples were heated from 10° C. at 10 C/min to 90° C. and then cooled down to 10° C. at 10 C/min. The crystalline CBD exhibited a thermal transition peak (melting point) at ca. 68° C.

The respective powder (solid) formulations were measured equally using ca. 8-10 mg of the respective sample.

Then the peak area of the respective sample is referenced against the peak area of the external standard to calculate the amount of crystals respectively the percentage of crystallinity in the respective samples.

All particle sizes were determined using a Beckmann Coulter, Delsa Nano S (Dynamic light scattering).

The bioavailability was tested using a Caco-2 model as further detailed below

FIG. 1: DSC thermogram of the solid formulation of example 2 (y-axis: heat flow (normalized) (W/g); x-axis Temperature (° C.))
solid line: t=0 (directly after production)
dashed line: after storage for 12 months at 5° C.
dotted line: after storage for 12 months at 25° C.
dashed-dotted line: after storage for 12 months at 40° C.

The DSC analysis showed no crystallization peaks after storage even at low temperatures illustrating the excellent stability of the solid formulation according to the present invention.

### Example 1

(i) For the preparation of the lipid phase, a total of 95 g of CBD was dispersed in 40.1 g of MCT at 75° C. under stirring until complete dissolution.

The lipid phase was kept warm until completing dispersion into the aqueous phase.

(ii) For the preparation of the aqueous phase, a total of 123 g pork gelatin, bloom 140, was hydrated and dissolved in 167.5 g warm water (40° C.). Subsequently, 23.5 dried glucose syrup, DE2023 was added and the solution was heated to process temperature of 75° C.

(iii) The warm aqueous phase was stirred, and the warm lipid phase was slowly added and homogenized at an elevated temperature (ca. 75° C.) to form a liquid formulation. No crystals were observed in the liquid formulation (visual assessment).

After viscosity adjustment with water (ca. 440 g), the emulsion was spray dried at ca. 180° C. inlet temperature, 80° C. outlet temperature using maltodextrin as carrier (approx. 6%).

A fine flowing powder was obtained, containing ca. 2.6% residual water and 31.9% CBD with >99% of the CBD in non-crystalline form as determined by DSC.

### Example 2

(i) For the preparation of the lipid phase, a total of 95 g of CBD was dispersed in 40.1 g of MCT at 75° C. under stirring until complete dissolution. Subsequently, 1.49 g of Vitamin E TPGS was added to the warm (75° C.) CBD/MCT mixture and homogenized.

The lipid phase was kept warm until completing dispersion into the aqueous phase.

(ii) For the preparation of the aqueous phase, a total of 123 g pork gelatine, bloom 140, was hydrated and dissolved in warm water (40° C.). Subsequently, 31.5% dried glucose syrup, DE2023 was added and the solution was heated to process temperature of 75° C.

(iii) The warm aqueous phase was stirred, and the warm lipid phase was slowly added and homogenized for 40 min at elevated temperature of ca. 75° C. to form a liquid formulation. No crystals were observed in the liquid formulation (visual assessment).

After viscosity adjustment with water, the emulsion was spray dried at ca. 180° C. inlet temperature, 80° C. outlet temperature using maltodextrin as carrier (approx. 6%).

A fine flowing powder was obtained, containing ca. 2.6% residual water and 31.9% CBD with >99% of the CBD in non-crystalline form as determined by DSC (i.e. below limit of detection (bld).

### Example 3: Bioavailability

CBD powder (solid) formulations (beadlets) comprising approx. 12-16 wt.-% of CBD and a particle size distribution of approx. 200 nm using different oils as outlined below were prepared to compare the influence of the oil on the bioavailability of CBD. The liquid formulations were prepared in analogy to example 1 above. Afterwards the respective beadlets were prepared by spraying the liquid formulation into a fluidized bed using native corn starch. The amount of the respective oil was chosen such to assure full dissolution of the CBD in the oil.

TABLE 1

| | Ex. 3 | Ref. 1 | Ref. 2 | Ref. 3 |
|---|---|---|---|---|
| Ingredient | [g] | | | |
| CBD | 30.7 | 30.8 | 29.6 | 33 |
| MCT | 14.32 | | | |
| Monoolein | | 57.22 | | |
| Monolinolein | | | 57.2 | |
| Corn Oil | | | | 61.2 |
| OSA starch (Cleargum COA1) | | 102 | 120 | 96 |
| OSA starch (Capsule HS) | 104.5 | | | |
| Glucose sirup | 55 | 25.5 | 30 | 10.2 |
| Water | 198 | 154 | 181.2 | 61.2 |
| CBD content liquid | 8 | 8 | 7 | 13 |
| formulation* [%] | | | | |
| CBD content solid | 15 | 14 | 12 | 16 |
| formulation* [%] | | | | |
| Crystals in solid | <1 | <1 | <1 | <1 |
| formulation (DSC) [%] | | | | |

The bioavailability was tested using Caco-2 Bioavailability assay as outlined below:

### Cell Culture Preparation

Caco-2 ECACC 86010202 (European Collection of Cell Cultures, Salisbury, UK) were cultured at 37° C., in atmosphere of 5% CO₂ in DMEM medium supplemented with 4.5 g/L D-Glucose, 4 mM L-Glutamine, 1 mM Sodium Pyruvate, 1% MEM Non-Essential Amino Acids, 50 μg/mL Gentamicin (Life Technologies Europe B.V., Zug, Switzerland) and 10% heat-inactivated FBS (Sigma-Aldrich, Buchs, Switzerland). Sub-confluent cells were tryptinized using 0.25% Trypsin/EDTA (Life Technologies Europe B.V., Zug, Switzerland).

### Caco-2 Bioavailability Assay

Cells were seeded at a density of 70,000 cells/well in 12-well cellQART® cell culture insert, PET membrane, 0.4 μM pore size, cell growth area 1.1 cm²/well (SABEU, Northeim, Germany). Media was changed every second to third day. After 21 days in culture, the barrier integrity of the differentiated cell monolayers grown on insert plates was confirmed by measuring transepithelial electrical resistance (TEER) using an EVOM2 Voltammeter (World Precision Instruments, Berlin) equipped with STX2-PLUS Electrodes. The TEER values correlate with the tightness of the confluent monolayer.

After 21 days the insert plates were washed 2 times with HBSS solution (HBSS pH 7.4 with Ca2+ and Mg2+, containing 5.5 mM D-(+)-glucose, Sodium Bicarbonate, and supplemented with 4 mM L-glutamine and 20 mM HEPES, Life Technologies Europe B.V., Zug, Switzerland) and incubated for 1 h at 37° C. in the CO2 incubator. The CBD treatment solutions were prepared in HBSS solution at a concentration of 15 M. The tratment solution was applied on the apical chamber (200 μL) and 1.5 mL of 4% BSA in HBSS solution were added to the basolateral chamber. The plate was then incubated for 3 h at 37° C. in the CO2 incubator on an orbital shaker (80 rpm).

After incubation the buffer solutions from the basolateral (BL) and the apical (API) compartments were collected and diluted in acetonitrile for analysis. The cell layers (CL) were washed once with HBSS solution, then 500 μL acetonitrile were added to the apical part. The cells were scraped off from the membrane using a pipet tip, and then collected for analysis. Cannabidiol was quantitated by a stable isotope dilution LC-MS method using an Agilent 1290 Infinity II UHPLC connected to a Bruker Impact II Q-TOF mass spectrometer. Eight cannabidiol calibration solutions covering a concentration range of 2.5 ng/ml to 2000 ng/ml were prepared in acetonitrile. A 500 ng/ml solution of deuterium labelled d3-Cannabidiol (CAS No. 1435783-16-6) in acetonitrile served as internal standard. Before injection, 250 ml of calibration solution was mixed with 25 ml of internal standard. Likewise, 250 ml of the centrifuged CaCo2 compartment samples were combined with 25 ml internal standard. The analytical column was a Raptor ARC C18 column (2.1×150 mm). The mobile phase was water/acetonitrile 24:76 (v/v) containing 5 mM ammonium formate and 0.1% v/v formic acid. The chromatogram was developed isocratically at a flow rate of 0.4 ml/min and a column temperature of 30° C. The injection volume was 1.5 ml. The column effluent was introduced into a VIP-HESI source (Bruker Daltonik GmbH) operating in positive ionization mode. The mass spectrometer was operating in full-scan mode scanning the range m/z 100-1000 with a spectrum rate of 4 Hz. Extracted high-resolution chromatograms at m/z 315.2319 and m/z 318.2507 with 5 mDa width were used for quantitation. A calibration curve was established by least squares regression by plotting peak area ratios (area analyte versus area internal standard) against added concentrations. Regression and computation of quantitative data was performed with TASQ 1.4 software (Bruker Daltonik). The amounts of CBD measured as a percent of the original CBD content from the apical compartment (API), basolateral (BL) compartment, and the cell layer (CL) are shown in Table 2. Cell permeability (reflecting bioavailability) of CBD is determined by the sum of the amounts measured in the basolateral compartment and the cell layer (BL+CL). Higher recovery indicates higher cell permeability of CBD.

TABLE 2
Results bioavailability test

| Oil | CBD in BL & CL* [%] |
|---|---|
| MCT | 41.4 |
| Monoolein | 23.8 |
| Monolinolein | 16.2 |
| Corn Oil | 23.2 |

Total amount of CBD in API, BL and CL was set to 100%.

As can be retrieved from table 2, the product form with the MCT oil exhibited a significantly better bioavailability compared to the other oils as the highest amount of CBD was detectable in the Cell lysate (CL) and Basolateral part (BL).

### Example 3: Pharmaceutical Compositions Comprising Solid Formulations According to the Present Invention

Tablets and capsules using the ingredients are prepared using the solid the solid formulations of examples 1, 2 and 3, respectively according to standard methods in the art.

TABLE 6

| | | |
|---|---|---|
| 1 | 2 | 3 |

| | |
|---|---|
| Example 5a: Tablets | Weight (mg) |

| | | | |
|---|---|---|---|
| Microcrystalline cellulose | 186 | 680 | 670 |
| (e. Vivapur 102) | | | |
| Cross-linked polyvinylpyrrolidone | 2 | 8 | 12 |
| (e.g. Kollidon CL) | | | |
| Magnesium Stearate | 1 | 8 | 12 |
| Silica | 1 | 4 | 6 |
| Solid formulation according to | 10 | 100 | 500 |
| example 1, 2 and 3 respectively | | | |
| Total | 200 | 800 | 1200 |

TABLE 7

| | | |
|---|---|---|
| 1 | 2 | 3 |

| | |
|---|---|
| Example 5b: Orally dispersable tablets | Weight (mg) |

| | | | |
|---|---|---|---|
| Mannitol (e.g. Pearlitol Flash) | 325 | 322 | 430 |
| Sorbitol (e.g. Neosorb P 200 SD) | 75 | 90 | 120 |
| Solid formulation according to | 100 | 188 | 250 |
| example 1, 2 and 3 respectively | | | |
| Total | 500 | 600 | 800 |

TABLE 8

| | 1 | 2 | 3 | 4 | 5 |
|---|---|---|---|---|---|
| Example 5c. Capsule fill | Weight (mg) | | | | |
| Microcrystalline cellulose | 54 | / | / | / | 34 |
| (e. Vivapur 102) | | | | | |
| Sorbitol | 5 | 17.5 | / | / | / |
| Mannitol | / | | 17.5 | / | / |
| Sodium starch glycolate | / | / | / | 26.25 | 12.5 |
| Silica | 1 | 3.5 | 3.5 | / | 3.5 |
| Solid formulation according to | 40 | 329 | 329 | 323.75 | 200 |
| example 1, 2 and 3 respectively | | | | | |
| Total | 100 | 350 | 350 | 350 | 250 |

1. A solid formulation comprising
(i) 5-85 wt-%, based on the total weight of the solid formulation, of at least one cannabinoid, and
(ii) 2 to 25 wt-%, based on the total weight of the solid formulation, of at least one MCT oil, and
(iii) 1 to 50 wt-%, based on the total weight of the solid formulation, of at least one emulsifier, and
(iv) optionally up to 40 wt-%, based on the total weight of the solid formulation, of at least one auxiliary agent, and
(v) less than 5 wt-%, based on the total weight of the solid formulation, of water,
wherein at least 50 wt-%, based on the total weight of the cannabinoid, of the at least one cannabinoid is in a non-crystalline form and
the ratio of the at least one cannabinoid to MCT oil is 2:1 to 4:1.

2. A solid formulation according to claim 1, wherein the formulation comprises
(i) 10 to 50 wt-%, based on the total weight of the solid formulation, of at least one cannabinoid, and
(ii) 3 to 20 wt-%, based on the total weight of the solid formulation, of at least one MCT oil, and
(iii) 0 to 7.5 wt.-%, based on the total weight of the solid formulation, of Vitamin E TPGS, and
(iv) 15 to 50 wt-%, based on the total weight of the solid formulation, of at least one emulsifier (other than the Vitamin E TPGS), and
(v) 1 to 40 wt.-%, based on the total weight of the solid formulation, of at least one auxiliary agent, and
(vi) less than 5 wt-%, based on the total weight of the solid formulation, of water,
wherein at least 50 wt-%, based on the total weight of the cannabinoid, of the at least one cannabinoid is in a non-crystalline form, and
wherein the ratio of the at least one cannabinoid to the MCT oil is 2:1 to 4:1.

3. The solid formulation according to claim 2, wherein the formulation consists essentially of the ingredients (i) to (vi).

4. The solid formulation according to claim 1, wherein the amount of the at least one cannabinoid is selected in the range from 20 to 45 wt.-%, preferably in the range from 30-45 wt.-%, based on the total weight of the solid formulation

5. The solid formulation according to claim 1, wherein the amount of the at least one MCT oil is selected in the range from 3 to 15 wt.-%, preferably in the range from 5 to 15 wt.-%, based on the total weight of the solid formulation.

6. The solid formulation according to claim 1, wherein at least one cannabinoid is chosen from the group consisting of cannabidiol, tetrahydrocannabinol, cannabinol, cannabigerol, cannabichromene, cannabicitran, cannabinodivarin, cannabiripsol, hexahydrocannabinol, delta-9-tetrahydrocannabiphoral, tetrahydrocannabivarin, endocannabinoid and endocannabinoid, preferably the at least one cannabinoid is cannabidiol.

7. The solid formulation according to claim 1, wherein the MCT oil is chosen from the group consisting of caproic acid, caprylic acid, capric acid and lauric acid.

8. The solid formulation according to claim 1, wherein the at least one emulsifier is chosen from the group consisting of modified (food) starches, gelatine and a mixture of gelatine and Vitamin E TPGS, preferably the emulsifier consists of a mixture of gelatine and Vitamin E TPGS.

9. The solid formulation according to claim 1, wherein the solid formulation comprises at least one auxiliary agent chosen from the group consisting of dyestuffs, thickeners, fillers, binders, flavours, antioxidants and pH buffer, preferably from the group consisting of glucose sirup, maltodextrin, corn starch and ascorbic acid.

10. The solid formulation according to claim 1, wherein the solid formulation is a spray dried solid formulation.

11. Use of a formulation according to claim 1 in food products, feed products, dietary supplements, pharmaceutical products, and personal care products.

12. A food product, feed product, dietary supplement, pharmaceutical, and personal care product comprising a formulation according to claim 1.

13. The pharmaceutical product according to claim 12, wherein the pharmaceutical product is in the form of a tablet, a capsule, an orally dispersable film, or a gummy.

14. Use of a MCT oil to enhance the bioavailability of at least one cannabinoid, preferably of cannabidiol, most preferably in the form of a solid formulation according to anyone of the embodiments 1 to 12.

15. A process for the preparation of a solid formulation according to claim 1, said process encompassing the steps of
(i) solubilising the at least one cannabinoid in a MCT oil, optionally followed by the addition of Vitamin E TGPS to form a lipid phase,
(ii) solubilising the at least one emulsifier (other than Vitamin E TGPS) and optionally one or more of the auxiliary agents in the water to form an aqueous phase, followed by
(iii) emulsification of the lipid phase into the aqueous phase, followed by
(iv) drying the emulsion, preferably by spray drying, even more preferably using maltodextrin as auxiliary (carrier) agent.
